(19) United States
(12) Patent Application Publication
(10) Pub. No.: US 2017/0241870 A1
(43) Pub. Date: Aug. 24, 2017

(54) Open-Cell Foam Environmental Indicator, and Methods of Use

(71) Applicant: Scott C. Smith, Osterville, MA (US)

(72) Inventor: Scott C. Smith, Osterville, MA (US)

(21) Appl. No.: 15/454,626

(22) Filed: Mar. 9, 2017

Related U.S. Application Data
(63) Continuation-in-part of application No. 15/402,335, filed on Jan. 10, 2017.
Application No. 15/402,335 is a continuation of application No. PCT/US2015/039905, filed on Jul. 10, 2015.
(60) Provisional application No. 62/306,982, filed on Mar. 11, 2016.
(60) Provisional application No. 62/022,760, filed on Jul. 10, 2014.

(51) Int. Cl. G01N 1/16; G01N 1/22; G01N 33/18; C08J 9/36

(52) U.S. Cl. CPC G01N 1/16; C08J 9/36; G01N 1/2273; G01N 33/1833; G01N 33/1826; G01N 33/1813; C08J 2205/05; C08J 2375/04; C08J 2323/02

(57) ABSTRACT

A method of removing and detecting the presence of substances from at least one of a body of water and the air. The method includes placing into the body of water or into the air an open-cell foam material, removing separate portions of the open-cell foam material from the water or air at different exposure times after the open-cell foam material was placed into the water or air, and determining the presence in the removed separate portions of one or more substances that were removed from the water or air by the open-cell foam material.

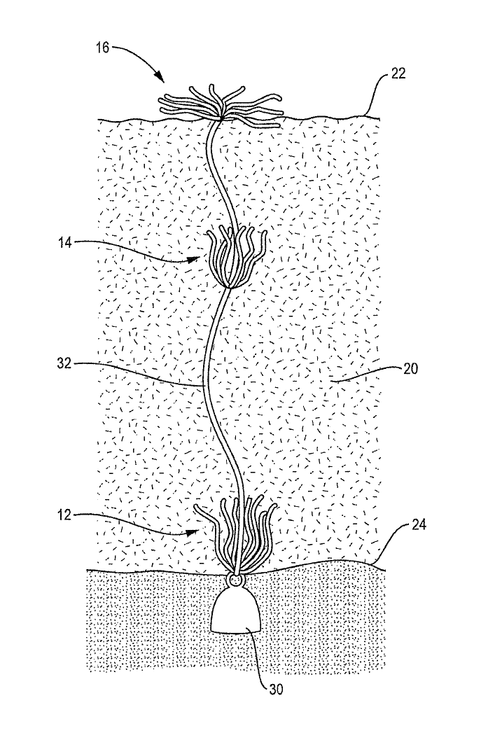

### CROSS-REFERENCE TO RELATED APPLICATIONS

This application claims priority of Provisional Application 62/306,982, filed on Mar. 11, 2016, the disclosure of which is incorporated herein by reference. This application also claims priority of and is a continuation in part of patent application Ser. No. 15/402,335, filed on Jan. 10, 2017, which itself is a continuation of and claimed priority of International Patent Application Number PCT/US15/39905, filed on Jul. 10, 2015, which claimed priority of U.S. Provisional Patent Application Ser. No. 62/022,760 filed on Jul. 10, 2014. The entire contents of each of these applications, including any Appendices, are incorporated by reference herein in their entireties.

### BACKGROUND

This disclosure relates to testing water and air for contaminants, and/or removal of contaminants from water or air (i.e., remediation).

The significance of trace chemicals in our water and air are of increasing interest due to their known and unknown effects on human health, as well as the health of animals and plants, and effects on the ecosystem. Animals and humans that are exposed to contaminants can absorb the chemicals into and through the skin, by breathing the air, and by drinking water including inadvertently while swimming in bodies of water effectively “concentrating” the chemicals into the body or skin. It is desirable to have an accurate and cost efficient method to analyze water and air for the presence of oil and other contaminants, including but not limited to diesel range organics, gasoline range organics, polychlorinated biphenyls (PCBs), fertilizers, pesticides, pharmaceuticals, organometals, metals, metalloids, volatile organic compounds (VOCs), semi-volatile organic compounds (SVOCs) and radioactive materials.

Traditional sampling by collecting and analyzing a split second “grab sample” is useful but has several limitations. Among those limitations is the inability to detect transient contaminants that are discharging sporadically and diffusing through the water column on an irregular basis, and the limited sample size that may contain only undetectable amounts of contaminants that are present at low concentrations and/or below the minimum detection level. Also, grab samples, by their nature, are instantaneous or reflect what is in the water for that split second. As a fish does not swim in the water for a split second and neither does a child, it is desired to have a sampling process that involves accumulation of contaminants over time in the same way that life forms are exposed to contaminants over time throughout the entire water column.

Chemicals and contamination are never in equilibrium in water, let alone water that is constantly flowing with many other variables to consider. Instantaneous/grab sampling reflects what is in the water for a split second, assumes the water being tested is in equilibrium, and does not take into consideration conditions like the constantly changing mixture of fresh water to the chemicals of concern when the grab sample is taken.

Semi-permeable membrane (SPMD) non open-cell foam sampling is the industry standard method for testing accumulation of contaminants due to the ability to concentrate the contaminant over an extended time period, typically 30 days. SPMDs are typically comprised of solid crystalline polyolefins. SPMD devices are described in U.S. Pat. Nos. 5,098,573 and 5,395,426. However, SPMDs are relatively expensive and as the molecular weight (size) of the contaminant increases, the absorption is reduced or eliminated. Furthermore, with the increase in diluted bitumen (“tar sands”) oil wastewater being used to irrigate crops, and of bakken oils, there is a need to monitor and detect the water column and produced water and/or wastewater used in irrigation and/or other applications which cause exposure risk for humans and the environment for drilling fluids, fracking fluids, chemicals used in the transportation of these “newer” oils, heavier oils, high molecular weight contaminants, some of which have specific gravities exceeding that of water, along with other contaminants. Some such contaminants can be difficult and cost prohibitive to monitor with SPMDs.

### SUMMARY

One subject of this disclosure is an open-cell foam material. The foam material can be produced from a copolymer of ethylene and alkyl acrylate, e.g., ethylene methyl acrylate (EMA). The open-cell structure behaves as the alveoli of the human lungs in that it maximizes surface area which maximizes the efficacy of the open-cell foam's ability to attract oil, chemicals, and related contamination at the molecular level, while repelling water.

One subject of this disclosure is an open-cell polyurethane (PU) foam matrix material. The polyurethane foam material has an open-cell structure that behaves as the alveoli of the human lungs in that it maximizes surface area, which maximizes the efficacy of the open-cell polyurethane foam's ability to attract oil, chemicals, and related contamination at the molecular level, while repelling water. The PU foam material preferably but not necessarily is highly open, with at least about 70%, and potentially at least about 80%, open cells, with the remainder of the cells closed. More preferably, the PU material comprises from about 70% up to about 100% open cells. The open cells can be created partially or fully in the polymerization of the PU, and/or can be enhanced by physical processes such as crushing to open closed cells. The greater the percentage of open cells, the greater the number of cells exposed to contaminants, and thus the greater the surface area of the PU material that is exposed to and thus can absorb/adsorb, contaminants. Also, the PU material can have a small cell size (in one non-limiting example, there are about 1,500 cells per square inch, which equates to about 2.3 cells per square millimeter, or an average cell size of about 0.43 mm (obviously, dependent on the shape of the cells)). Smaller cells create more surface area per volume as compared to larger cells. Thus, smaller cells lead directly to greater cell wall area that can be used to take up contaminants. A material such as the PU material, with a large percentage of open cells, having a small cell size, thus can greatly increase the surface area of material that is exposed to contaminants, and thus can more readily take-up the contaminants, and can absorb a larger quantity of contaminants.

Open-cell polyurethane foam (and other foams disclosed herein) cumulative testing as described herein identifies what is in the water over time. It can also allow the calculation of the total amount of oil or other contaminants recovered from the water, e.g., after an oil spill. Such testing can use (but need not use) EPA testing procedures. This cumulative testing has much less variability than instantaneous grab samples when it comes to potential dilution of the water body being tested and variability within the body of water and various connections to the body of water being tested. It has been established that grab sample testing exhibits such variability that it can lead to non-detects when chemicals of concern are actually present. When considering how animals and humans are exposed to various bodies of water from the bathtub/shower at home, to swimming in a lake, pond, or ocean, the concentration index of the open-cell polyurethane foam matrix cumulative testing appears to be a more accurate representation of the chemicals of concern in the water, especially when the body of water is not in equilibrium. Some of the more dangerous chemicals of concern can enter the body through the skin; the skin attracts these chemicals and “concentrates” the chemicals in the human body.

In one example, the open cell foam material can be composed of one or more polyolefins, including but not limited to polyolefin elastomers. In another example, open-cell polyurethane foam structures provide high surface area due to the interconnected structure and open nature of the individual cells. The oleophilic nature of the polyurethane inhibits the absorption of water and promotes absorption and adsorption of oils and related substances.

In one example, the cumulative environmental indicator device (or detector) can be fabricated from a very specific formulation of an open-cell foam. Specifically, this foam is produced from 80-100% ethylene acrylate copolymer. Blends of LDPE can be used also. One embodiment/formulation of this open-cell foam is described in U.S. Pat. No. 8,853,289, the disclosure of which is incorporated herein by reference. Another embodiment/formulation of this open-cell foam is described in US patent application publication US 2013/0240451 A1, the disclosure of which is incorporated herein by reference.

While open-cell polyurethane is one preferred material for the open-cell foam discussed herein, what is contemplated herein includes any open-cell foam (with at least some of the cells open), and produced from one or more polymers, such polymers including but not limited to EMA, ethylene vinyl acetate (EVA), ethylene-ethyl acrylate (EEA), ethylene-butyl acrylate (EBA), ethylene propylene diene monomer (EPDM), elastomers, polyolefin elastomers, low density polyethylene (LDPE), linear low density polyethylene (LLDPE), high density polyethylene (HDPE), polypropylene (PP), neoprene, styrene butadiene rubber, ionic copolymers, other synthetic rubbers, natural rubber, chlorinated polyethylene (CPE), olefin block copolymers, ethylene maleic anhydride copolymer, very low density polyethylene (VLDPE), singe site initiated polyolefins, metallocene catalyzed polyolefins, silane-modified polymers (including but not limited to silane grafted, silane functionalized, and silane cross-linked polymers), maleic anhydride grafted polymers, styrene-butadiene-styrene copolymers, polyisoprene, and equivalents to any and all of these polymers. Silane modification of polymers can occur during the manufacturing process of the open-cell foam, or as a separate step after the foaming process, e.g., the silane can be applied in liquid form post-foaming. Further, specific silane-modified polymers may be tailored to target specific contaminants, such as VOCs and SVOCs related to oils and other petroleum products, and surfactants, including but not limited to methylene blue active substances (MBAS).

One preferred foam density is in the range of from about 1.0 pcf (pounds per cubic foot) to about 50.0 pcf, but the foam can be any density, with preferred densities less than the specific gravity of water (62.3 pcf at 70° F.).

The open-cell foam can be produced in any convenient manner. It can be extruded, including but not limited to roll form, molded in a closed or open mold, or produced in a bun/batch process, for example. The open-cell foam can be crosslinked or non-crosslinked. Also, the open-cell foam can be manufactured using either physical blowing agents or chemical blowing agents. Furthermore, a bio-degradable initiator may be added to the foam so that after use it will degrade over time in a landfill environment when disposed.

The polyurethane foam (and the other foams) are preferably highly oleophilic. Depending upon target contaminants, this disclosure also contemplates formulations with varying levels of polarity for detecting radioactive materials/metals along with other chemicals/contaminants that tend to have increased polarity. Its open-cell structure allows for absorption of oils and other lipids into the network cell structure of the foam. Preferred but non-limiting cell size ranges are from about 0.1 mm to about 3.5 mm. Water is repelled. This selective absorption of lipids makes the polyurethane foam (and the other foams) desirable for use as an oil and related chemicals indicator/detector, and for remediation, in bodies of water.

A cumulative environmental indicator device herein can comprise one or more separate portions (e.g., pieces or strips) of the subject polyurethane foam (or the other foams) that are held in a water column, at one depth or at multiple depths. The foam is preferably but not necessarily less dense than water so strip(s) will extend upward in the water column from the location where they are held/anchored. Each strip can extend over some or all of the depth (i.e., the height of the water column). The foam is left in place for an extended period of time, longer than is accomplished with a grab sample. The time can be any amount of time—seconds, minutes, hours, days, weeks or months, depending on the situation and desired results. The foam absorbs and adsorbs oils and other contaminants. These contaminants are accumulated by the foam. The foam is then removed and tested for the presence and concentration of contaminants. Since the indicator can span different depths, the results can determine the presence and concentration of one or more contaminants at different depths of the water column, from the surface to the bottom, as desired. Also, as the foam portions can be placed at different locations in a body of water (as well as at different depths if desired), they can detect the movement of plumes of contamination through the water.

The indicator can be fabricated into a number of structures to suit the application of the indicator. One preferred structure is an assembly of strips, typically 0.5-0.75 inch×0.5-0.75 inch×12-18 inches. The strips can be fastened together tightly at the center to form a structure with multiple “fingers” or “blades.” This structure exposes a large surface area to the environment, and allows flow through (between the fingers of) the indicator. These indicators are then fastened to a rope line or similar tether with a weight at one end, and are submerged into the water body, leaving indicators at various depths. Another alternative is to have strips of the foam that are anchored to the bottom and extend to the surface, over the entire water column; this is called “eelgrass” since it looks like eelgrass that grows in the ocean. Other forms can include strips and smaller cubes and pieces in other shapes. Another form includes a structure with multiple smaller fingers sized such that the structure can be cast (either weighted or not weighted) into the water or water column, e.g., with a fishing rod, and retrieved. Placing the open-cell foam material into the body of water may comprise dragging the open-cell foam material behind a boat that moves through the water, or floating the open-cell foam material on the surface of the water, or coupling the open-cell foam material to a dock, or placing the open-cell foam material in a bathtub or sink. Any form can be placed anywhere in the water column and/or on the surface of the water. Smaller pieces can be held in place in nets or other containers with holes to allow water flow. Such containers can be made of plexiglass or other materials.

The indicator can be designed to monitor the water for accumulation of chemicals over time. The indicator can also be used to remove the chemicals from the water (i.e., remediation). Indicators can be in the forms of eelgrass, cubes, pieces, and/or strips, or other forms, and can be but need not be contained in a cylinder or net. These forms can be floating on the surface or suspended and/or submerged in the water column, e.g., using anchors.

Indicators that are fabricated from the described polyurethane foam (and the other foams) can detect and remove organic species and other substances in bays and harbors as well as open bodies of water and fresh water lakes, rivers, and streams, and in residences (e.g., bathtubs and sinks).

Advantages of this indicator are its efficient cost, ease of deployment, durability during deployment and in use, and ability to collect large samples over an extended time period.

Upon retrieval of the indicator from the water or air, the open-cell foam can be placed into a sealed container and sent to a qualified lab to test the open-cell foam with various EPA and other testing methods.

Furthermore, the foam can remove and detect food sources for what is known as Blue-Green Algae/Cyanobacteria, including phosphorous. By deploying various designs of fabricated open-cell foam it is contemplated that phosphorous can be effectively removed to assist in the prevention of Blue-Green Algae/Cyanobacteria blooms. One such embodiment is a solar powered filtration system that uses open-cell foam pieces (e.g., cubes) in a flow-through container (filter) such as a cylindrical structure that is scalable to the size of the body of water. A pump can be used to flow water through the filter. The pump can be but need not be solar or battery powered. The filter media (polyurethane or other foam pieces) can be changed as necessary. This filtration system can be used to remove all other substances that are referenced in this disclosure, in various bodies of water.

In one aspect, a method of removing and detecting the presence of substances from at least one of a body of water and the air includes placing into the body of water or into the air an open-cell polyurethane or other foam material, removing separate portions of the open-cell foam material from the water or air at different exposure times after the open-cell foam material was placed into the water or air, and determining the presence in the removed separate portions of one or more substances that were removed from the water or air by the open-cell foam material.

Embodiments may include one of the following features, or any combination thereof. The open-cell foam material may be substantially non-polar. The open-cell foam material may comprise a copolymer comprising a polar component. The substances that are removed may be selected from the group of substances consisting of oil, diesel range organics, gasoline range organics, drilling fluids, biocides, glutaraldehyde, metals, organometals, metalloids, VOCs, SVOCs, pesticides, PCBs, radioactive substances, fertilizers, solvents, human waste, pharmaceuticals, and components thereof.

Embodiments may include one of the following features, or any combination thereof. Placing the open-cell foam material may comprise suspending a plurality of separate structures at different levels through a height of a water column. Placing the open-cell foam material may further comprise placing a plurality of separate structures at different locations in the body of water. Placing the open-cell foam material may further comprise floating a structure at least partially on the surface of the water. Placing the open-cell foam material into the body of water may comprise casting the open-cell foam material into the water with a fishing rod. Placing the open-cell foam material into the body of water may comprise dragging the open-cell foam material behind a boat that moves through the water, or floating the open-cell foam material on the surface of the water, or coupling the open-cell foam material to a dock, or placing the open-cell foam material in a bathtub or sink. Removing separate portions of the open-cell foam material from the water or air at different exposure times after the open-cell foam material was placed into the water or air, can take place at more than one time over an exposure time of at least eight hours, or at least one day.

Embodiments may include one of the following features, or any combination thereof. The open-cell foam material may comprise a plurality of separate structures selected from the group of structures consisting of strips, strips that are longer than a height of a water column, cubes, and small pieces. The separate structures may be held in place by one or more of an anchor, a weight, a netting, and a container with openings to allow the flow of water therethrough.

In another aspect, a method of removing and detecting the presence of substances from at least one of a body of water and the air, includes suspending a plurality of separate structures comprising an open-cell foam material in the form of strips, strips that are longer than a height of a water column, cubes, and small pieces, at different levels through a height of a water column of the body of water and at different locations in the body of water, removing separate portions of the structures from the water at different exposure times after the structures were placed into the water, and determining the presence in the removed separate portions of one or more substances that were removed from the water by the structures, wherein the substances are selected from the group of substances consisting of oil, diesel range organics, gasoline range organics, drilling fluids, biocides, glutaraldehyde, metals, organometals, metalloids, VOCs, SVOCs, pesticides, PCBs, fertilizers, solvents, human waste, pharmaceuticals, and components thereof.

In one aspect of the disclosure when testing air, the open-cell foam can be moved through the air (e.g., flown through the air with a drone or helicopter) and then tested for contaminants as described elsewhere herein. Or the foam can be placed in the air at one or more locations on the ground and/or at different heights above the ground, and left for desired amounts of time (such as described herein relative to water testing).

### BRIEF DESCRIPTION OF THE DRAWING

The drawing depicts one non-limiting example of the placement of open-cell polyurethane (PU) foam material into a body of water.

### DETAILED DESCRIPTION OF EXAMPLES

Methods of removing and detecting the presence of substances (such as, but not limited to, contaminants) from a body of water or the air are disclosed. As a first step, an open-cell polyurethane foam material (or other foam materials, as described elsewhere herein) can be placed into the body of water or into the air. The placement can be at one or more locations in the body of water or air, and at one or more depths or heights in the body of water or in the air. After desired exposure times, one or more separate portions of the open-cell foam material are removed from the water or air. The presence in the removed separate portions of one or more substances that were removed from the water or air by the open-cell foam material are then determined, typically by standard EPA testing procedures.

The methods are effective both to determine the presence of substances such as contaminants in the water or air, and also to remove such substances from the water or air. The methods thus can be used for contaminant detection and/or filtration or remediation.

The drawing depicts three groups of strips or “blades” of open-cell polyurethane foam material 12, 14 and 16. Each group has multiple strips that are held together at about their centers, The groups are fastened to a line 32 that is held on the bottom 24 of water body 20 by weight or anchor 30. In this example group 16 floats on the water surface 22, while groups 12 and 14 are held at different depths below the surface. This disclosure allows for the placement of open-cell foam material at any one or more heights of a body of water and/or the air, and at one or more locations in the body of water or air. Various non-limiting methods of exposing the open-cell material to water or air are described herein; any such method can be used as desired or as necessary depending on the body of water or the air mass, and/or the testing regime that is desired under the circumstances.

After desired exposure times, one or more separate portions of the foam material are removed from the water or air. This can be done by clipping or cutting a piece of foam, or removing an entire group or other portion or separate piece of foam, for example. The exposure times can be from seconds to minutes to hours to days to weeks to months, depending on the particular testing regime. Since the foam absorbs and adsorbs certain materials (described elsewhere herein), the removed portions of the foam can be tested for particular substances. The foam can act as an accumulator for these substances. Also, the different locations and different exposure times allow for a tailored review of contaminants, their locations and their movement within the water or air.

The subject materials have been used in extensive testing of various bodies of water, both salt and fresh water, including open bodies of water, rivers, streams and irrigation canals. Open-cell foam material has been shown to remove from water at least the following types of contaminants. Non-limiting examples of each type of contaminant are also listed; these examples illustrate contaminants that are in each type or group, but are not limiting.
Metals and Metalloids: examples include but are not limited to Arsenic, Barium, Boron, Cadmium, Cooper, Lead, Manganese, Mercury, Nickel, Phosphorus, Vanadium, Yttrium.
Volatile Organic Compounds (“VOCs”): examples include but not limited to Tetrachloroethane, Trimethylbenzene, Butanone, Acetone, Benzene, Ethylbenzene, Methylene Chloride, Xylene, Toluene.
Semi Volatile Organic Compounds (“SVOCs”): examples include but not limited to Naphthalene, Anthracene, Chrysene, Fluorene, Hexachlorobenzene, Nitrobenzene, Pyrene, Dimethyl Phthalate.
Drilling/Transportation/biocide fluids: examples include but not limited to Glutaraldehyde, Benzene compounds, Toluene compounds, Xylene compounds.
Coal related fluids: examples include but not limited to MCHM (4-Methylcyclohexanemethanol).
Fluids/Solvents that for example leach from landfills into peoples homes: examples include but not limited to 1,4 Dioxane.
Radioactive materials: examples include but not limited to: Strontium, Uranium, Yttrium, Rhenium.

### Test Methods and Results

Results of water testing using an environmental indicator made from an open-cell foam material of a type disclosed in the US patent that is incorporated by reference herein (e.g., 100% EMA), were set forth in the Appendices of the priority U.S. Provisional Patent Application Ser. No. 62/022,760 filed on Jul. 10, 2014. The entire contents of this Provisional patent application, including the Appendices, are incorporated by reference herein in their entirety. Accordingly, the data disclosed in the Appendices is also incorporated herein by reference. Some of that data is set forth and summarized below.

The testing methods used by certified third party laboratories include but are not limited to: EPA SW8015B, EPA SW7471A, EPA SW6010B, EPA SW8270C, EPA SW8260B, IH-004, ALS Method 8270 (for MCHM), and EPA 1664 (modified) for oil and grease.

One test set detected low levels of PCBs in the harbor at New Bedford, Mass. Data was included in the appendices.

In Knapp Creek, Pa., Oak Glen Nature Preserve, Ohio, Lynchburg, V A and Aliceville, Ala., it was discovered that the cumulative environmental indicator detected and removed organometals, metals, metalloids, VOCs, and SVOCs, including chemicals like 4-methylcyclohexanemethanol (MCHM), which was spilled in Charleston, W. Va., USA. See third party testing results (in the appendix), which illustrates the accumulation over time into the open-cell EMA foamed matrix, mimicking environmental uptake by living organisms. It is of note that arsenic, barium, lead, and mercury were detected in Knapp Creek. There were harsh winter weather conditions and the open-cell EMA retained and absorbed these metals and related compounds.

Results of uses of the environmental indicator in water are disclosed in the appendices I-V of the priority Provisional application, which are incorporated by reference herein in their entireties. A brief discussion of those appendices follows.

Appendix I of the priority Provisional application is a table (printed on two pages) that includes a comparison of the environmental indicator to a grab sample taken from the surface of the water (at 8:30 AM when the test began) after a coal ash spill in the Dan river in Eden, N.C. See tables 1A and 1B for the data. The indicators (environmental indicators or “EI”) were in three different forms: one was anchored with strips at the bottom, middle and top of the water column (such as in the drawing), a second was in the form of floating eelgrass, and the third was in the form of a mitt made of the material that was submerged in the water column. These various forms of indicators were removed at three different times to show exposure or accumulation of contaminants over time, and the samples were sent for analysis to a third-party lab. Organometals, metals, metalloids and SVOCs were detected at different times and different heights in the water column. Samples were taken with instantaneous grab samples along with open-cell foam cumulative samples at various exposure times. The grab sample showed non-detects (N/D) for everything with the exception of iron. The open-cell foam cumulative samples at various exposure times detected the presence of metals and SVOCs. Furthermore, the open-cell foam cumulative samples (indicators) illustrated the importance of sampling the entire water column at various depths. For example, the bottom indicator detected the presence of manganese, phosphorus, and titanium while the middle and top indicators showed non-detects. Also, the open-cell foam mitt that was exposed to the water near shore from top to bottom in about 2 feet of water for 3 minutes (right next to where the instantaneous grab sample was taken) detected the presence of iron, manganese, phosphorous, titanium, and SVOCs while the grab sample only detected the presence of iron.

Appendix II of the priority Provisional application is a table (printed on three pages) that includes results taken after a bakken oil spill in Aliceville, Ala. A variety of forms of the indicator were placed into the water in and around the site of this bakken oil train explosion and a variety of residual SVOCs and VOCs were detected leaching out of the soil and into the wetlands approximately three months after the oil train explosion. Pure oil samples were taken as leaching from the soil and these were used as a baseline to compare to what the indicators absorbed in the nearby surrounding waters from the wetlands.

Appendix III of the priority Provisional application is a table (printed on one page) that includes results taken from the banks of the James River in Lynchburg, Va. These samples were collected from the river bank of the James River after a bakken oil train exploded. An indicator was placed directly into the contaminated river bank and was then placed into a sealed glass jar and sent to a third-party lab. A variety of SVOCs, VOCs, and organometals, metals, and metalloids were detected that were consistent with prior bakken oil train explosions. Appendices III a of the priority Provisional application (four pages) and III b of the priority Provisional application (four pages) are publicly available results as posted by the James River Association and Arcadis. The James River Association and Arcadis tests showed non-detects which includes but is not limited to the following metals and oil compounds while the environmental indicators detected these metals and oil compounds: barium, chromium, nickel, phosphorus, vanadium, acetone, tri-methylbenzene, xylene and napthalene.

Appendix IV of the priority Provisional application is a table (printed on two pages) that includes results from various open-cell foam cumulative samples were taken from the water column after an oil spill in Galveston, Tex. See Table 4 for the data. Eelgrass indicators and submerged indicators were placed into the water. Of four samples taken (3 surface (two with eelgrass and one with an environmental indicator) and 1 middle of water column), the middle indicator detected chromium, cobalt, and lead were detected but not detected in the others. Also, vanadium was detected on the surface eelgrass after exposure of approximately 43 hours but had a non-detect after 20 hours. Vanadium was detected by the middle indicator in the middle of the water column after 20 hours (again there was a non-detect on the surface after 20 hours).

Appendix V of the priority Provisional application is a table (printed on one page) that includes results taken from three locations in Nantucket harbor, Nantucket, Mass. Environmental indicators were placed in the entire water column and retrieved at different lengths of time to monitor exposure over time. When retrieved, the indicators were placed in plastic and glass sealed containers for third party lab testing. At the boat dock, acetone was detected in the middle of the water column but not at the top or bottom. At the town pier, vanadium was detected at the bottom of the water column but not at the middle of the water column.

Various open-cell foam cumulative samples were taken in the Cawelo water district in Kern County, Calif. where wastewater from oil drilling and/or refining operations is filtered and diluted with fresh water and then feed to a canal system that is used to irrigate crops. Baseline oil-water as it comes out of the ground was tested along with downstream water throughout the canal system. See tables 2A-2D for data (blank cells in the tables are non-detects). TPH stands for total petroleum hydrocarbons, baseline oil is the subject oil that is leaking or spilling in its raw form, irr pond is an irrigation pond, Poso Creek was baseline oil-water coming right out of the ground in its raw state. The post-filtration dilution locations were various locations throughout the irrigation canal system in the Cawelo Water District of Kern County, Calif., USA. The presence of oil and VOCs were found downstream that matched the chemicals found in the baseline oil-water mixture. Also, there were various non-detects throughout the canal system which confirmed that the water is not in equilibrium and the importance of multiple sample points with the ability to detect chemicals over time/exposure.

Separately from the irrigation water in the Cawelo water district and in another part of Kern County, various open-cell foam cumulative samples were taken from the surface of oil wastewater discharge points in unlined pits. The data gathered (presented in Table 3) illustrates that even in this relatively confined water discharge system that there is no equilibrium of contaminants in water. All samples were exposed to the surface water for 30 minutes. Metals, metalloids, and VOCs showed various detects and non-detects (blank table cells), along with variation in concentration levels.

The fact that testing with the subject material has had non-detects and detects for chemicals of concern in the same bodies of water is not only expected but is absolute proof that chemicals/contamination are not in equilibrium in water. Thus, using an accumulator as described herein is highly beneficial for determining actual contaminants in water.

TABLE 1A

| | Exposure Time | | | | |
|---|---|---|---|---|---|
| | | Instan- | | | |
| | | taneous- | | 3 Hours/ | |
| | 3 Minutes | 1 Second | 9 Hours | Surface | 3 Hours |
| | Sample | | | | |
| | | Grab | | El Float | |
| | Mitt | Sample | El Bot | Kayak | El Mid |
| | Units | Units | Units | Units | Units |
| Metals | (ppm) | (ppm) | (ppm) | (ppm) | (ppm) |
| Iron | 830 | 0.81 | 480 | 220 | 110 |
| Manganese | 11 | N/D | N/D | 33 | N/D |
| Phosphorus | 17 | N/D | 13 | 13 | N/D |
| Titanium | 47 | N/D | 17 | N/D | N/D |
| | Units | Units | Units | Units | Units |
| SVOC's | (ppb) | (ppb) | (ppb) | (ppb) | (ppb) |
| Bis(2-ethylhexl) | 640 | N/D | 2200 | 270 | 230 |
| phthalate | | | | | |
| Di-n-octyl | N/D | N/D | 340 | N/D | 720 |
| phthalate | | | | | |

TABLE 1B

| | Exposure Time | | | | |
|---|---|---|---|---|---|
| | 3 Hours | 3 Hours | 6.5 Hours | 6.5 Hours | 6.5 Hours |
| | Sample | | | | |
| | El Top | El Bot | El Top | El Mid | El Bot |
| | Units | Units | Units | Units | Units |
| Metals | (ppm) | (ppm) | (ppm) | (ppm) | (ppm) |
| Iron | 110 | 210 | 120 | 220 | 310 |
| Manganese | N/D | N/D | N/D | N/D | 5.4 |
| Phosphorus | N/D | N/D | N/D | N/D | 15 |
| Titanium | N/D | N/D | N/D | N/D | 12 |
| | Units | Units | Units | Units | Units |
| SVOC's | (ppb) | (ppb) | (ppb) | (ppb) | (ppb) |
| Bis(2-ethylhexl) | 210 | 120 | 810 | 1500 | 430 |
| phthalate | | | | | |
| Di-n-octyl | 270 | 550 | 550 | N/D | N/D |
| phtalate | | | | | |

TABLE 2A

| | Exposure Time | | | |
|---|---|---|---|---|
| | Instant | Instant | 17 Hours | 17 Hours |
| | Location | | | |
| | Poso | Poso | Post | Post |
| | Creek | Creek | Dilution/ | Dilution/ |
| | Oil | Oil | Water | Water |
| | Field | Field | Treatment | Treatment |
| | Sample | | | |
| | Baseline | Baseline | Canal | Canal |
| | Oil w Mitt | Oil w/El | Top El | Bottom El |
| Oil | PPM | PPM | PPM | PPM |
| TPH C20-C34 | 240000 | 480000 | 940 | 340 |
| VOC's | PPB | PPB | PPB | PPB |
| Acetone | 440 | 530 | 57 | |
| 1,2,4- | 400 | 160 | | |
| Trimethlybenzene | | | | |
| 1,3,5- | 110 | 52 | | |
| Trimethlybenzene | | | | |
| m,p-Xylene | 120 | 66 | | |
| o-Xylene | 70 | | | |
| Methylene Chloride | 89 | 82 | 26 | |

TABLE 2B

| | Exposure Time | | | |
|---|---|---|---|---|
| | 30 Minutes | 30 Minutes | 5 Hours | 5 Hours |
| | Location | | | |
| | Post | Post | Post | Post |
| | Dilution/ | Dilution/ | Dilution/ | Dilution/ |
| | Water | Water | Water | Water |
| | Treatment | Treatment | Treatment | Treatment |
| | Sample | | | |
| | Irr Pond | Unlined | Canal | Canal |
| | Eelgrass | Pond El | Top El | Bottom El |
| Oil | PPM | PPM | PPM | PPM |
| TPH C20-C34 | 1300 | 180 | 270 | 130 |
| VOC's | PPB | PPB | PPB | PPB |
| Acetone | | | 79 | |
| 1,2,4- | | | | |
| Trimethlybenzene | | | | |
| 1,3,5- | | | | |
| Trimethlybenzene | | | | |
| m,p-Xylene | | | | |
| o-Xylene | | | | |
| Methylene Chloride | | 32 | 31 | 30 |

TABLE 2C

| | Exposure Time | | |
|---|---|---|---|
| | 7 Months | 30 Minutes | 30 Minutes |
| | Location | | |
| | Post Dilution/ | Post Dilution/ | Post Dilution/ |
| | Water | Water | Water |
| | Treatment | Treatment | Treatment |
| | Sample | | |
| | Canal Top | Canal Top | Canal Middle |
| | Indicator A | Indicator | Indicator |
| Oil | PPM | PPM | PPM |
| TPH C20-C34 | 230 | | |
| VOC's | PPB | PPB | PPB |
| Acetone | | | |
| 1,2,4- | | | |
| Trimethlybenzene | | | |
| 1,3,5- | | | |
| Trimethlybenzene | | | |
| m,p-Xylene | | | |
| o-Xylene | | | |
| Methylene Chloride | | 56 | 44 |

TABLE 2D

Exposure Time

| | |
|---|---|
| 30 Minutes | 44 Hours |

Location

| | |
|---|---|
| Post Dilution/ | Post Dilution/ |
| Water Treatment | Water Treatment |

Sample

| | Canal Bottom | |
|---|---|---|
| | Indicator | Canal Eelgrass |
| Oil | PPM | PPM |
| TPH C20-C34 | | |
| VOC's | PPB | PPB |
| Acetone | | |
| 1,2,4- | | |
| Trimethlybenzene | | |
| 1,3,5- | | |
| Trimethlybenzene | | |
| m,p-Xylene | | |
| o-Xylene | | |
| Methylene Chloride | 48 | 26 |

TABLE 3

| | Exposure Time | | |
|---|---|---|---|
| | 30 Minutes | 30 Minutes | 30 Minutes |
| | Location | | |
| | Kern County | Kern County | Kern County |
| | Sample | | |
| | Eelgrass A | Indicator | Eelgrass B |
| | PPM | PPM | PPM |
| Oil | | | |
| TPH C10-C20 | 320 | 650 | 9100 |
| TPH C20-C34 | 670 | 1900 | 27000 |
| Metals | | | |
| Barium | 11 | 10 | |
| Boron | 31 | 42 | 14 |
| Copper | | 25 | |
| Iron | 220 | | |
| Phosphorous | 12 | | |
| Sodium | 2000 | 3000 | |
| VOC's | PPB | PPB | PPB |
| Acetone | 250 | 560 | 90 |
| 1,2,4- | 65 | 46 | 240 |
| Trimethlybenzene | | | |
| 1,3,5- | | | 69 |
| Trimethlybenzene | | | |
| 2-Butanone | 36 | 75 | |
| Benzene | 76 | 360 | 31 |
| Carbon Disulfide | | | 31 |
| m,p-Xylene | 130 | 180 | 330 |
| n-Butylbenzene | | | |
| o-Xylene | 64 | 97 | 170 |
| Ethylbenzene | 36 | 63 | 120 |
| Methylene Chloride | 27 | | |
| Isopropylbenzene | | | 30 |
| Napthalene | | | 40 |
| N-Propylbenzene | | | 58 |
| Toluene | 200 | 600 | 240 |

TABLE 4

| | Exposure Time | | | |
|---|---|---|---|---|
| | 20 Hours | 19 Hours | 19 Hours | 19 Hours |
| | Sample | | | |
| | Eelgrass | Eelgrass | Indicator | Indicator/ |
| | Surface | Surface | Surface/Top | Middle |
| Metals | PPM | PPM | PPM | PPM |
| Aluminum | 16 | 221 | 76.4 | 226 |
| Barium | 1.57 | 5.29 | 2.31 | 2.97 |
| Calcium | 4960 | 4640 | 3580 | 4600 |
| Chromium | N/D | N/D | N/D | 0.594 |
| Cobalt | N/D | N/D | N/D | 0.641 |
| Lead | N/D | N/D | N/D | 0.69 |
| Magnesium | 886 | 1100 | 943 | 1140 |
| Manganese | 4.39 | 49.8 | 17 | 49.8 |
| Molybdenum | 0.514 | N/D | N/D | 0.725 |
| Iron | 54.3 | 0.677 | 248 | 857 |
| Sodium | 4330 | 5160 | 5070 | 5300 |
| Tin | 3.27 | 4.53 | 2.53 | N/D |
| Titanium | 0.493 | 4.67 | 1.66 | 4.07 |
| Vanadium | N/D | 0.776 | N/D | 0.757 |
| Zinc | 55.1 | 35.4 | 27.5 | 33.4 |

Results of water testing using an environmental indicator made from an open-cell polyurethane foam material compared to results testing the same water using a polyolefin foam material of a type disclosed in U.S. Pat. No. 8,853,289 are set forth in tables 5 and 6, below. In table 5, one inch cubes of the two materials were used, and in table 7, strips of the two materials were used. In each case, the volume of contaminated water (water contaminated with bakken oil), the volume of the material, and the exposure times, were the same. The level of contaminants (semi-volatile organic compounds (SVOCs) and (volatile organic compounds (VOCs) detected are expressed in parts per billion (ppb).

TABLE 5

| | 1″ cube | | |
|---|---|---|---|
| | Open-Cell | 1″ cube | |
| | Polyurethane | Polyolefin | Absorption |
| | (PU) Foam | (PO) Foam | Ratio |
| Contaminant (PPB) | PPB | PPB | PU/PO |
| SVOCs | | | |
| 1-Methylnaphthalene | 120,000 | 43,000 | 2.79 |
| 2-Methylnaphthalene | 130,000 | 46,000 | 2.83 |
| Phenanthrene | 17,000 | Not detected | |
| VOCs | | | |
| 1,2,4 Trimethylbenzene | 190,000 | 78,000 | 2.44 |
| 1,3,5 Trimethylbenzene | 39,000 | 15,000 | 2.60 |
| Benzene | 7,000 | Not detected | |
| Ethylbenzene | 50,000 | 15,000 | 3.33 |
| Isopropylbenzene | 30,000 | 11,000 | 2.73 |
| m,p Xylene | 140,000 | 47,000 | 2.98 |
| Naphthalene | 43,000 | 12,000 | 3.58 |
| n-Butylbenzene | 28,000 | 9,900 | 2.83 |
| n-Propylbenzene | 36,000 | 13,000 | 2.77 |
| o-Xylene | 84,000 | 31,000 | 2.71 |
| p-Isopropyltoluene | 22,000 | 8,500 | 2.59 |
| sec-Butylbenzene | 17,000 | 6,300 | 2.70 |
| Toluene | 64,000 | 13,000 | 4.92 |

TABLE 6

| | Open-Cell | Open-Cell | |
|---|---|---|---|
| | Polyurethane | Polyolefin | |
| | strips | strips | Absorption |
| Contaminant (PPB) | PPB | PPB | Ratio PU/PO |
| SVOC's | | | |
| 1-Methylnaphthalene | 47,000 | 15,000 | 3.13 |
| 2-Methylnaphthalene | 50,000 | 15,000 | 3.33 |
| Naphthalene | 15,000 | Not detected | |
| Phenanthrene | 9,100 | Not detected | |
| VOC's | | | |
| 1,2,4 | 65,000 | 10,000 | 6.50 |
| Trimethylbenzene | | | |
| 1,3,5 | 14,000 | 2,100 | 6.67 |
| Trimethylbenzene | | | |
| Ethylbenzene | 19,000 | 3,000 | 6.33 |
| Isopropylbenzene | 10,000 | 1,700 | 5.88 |
| m, p Xylene | 59,000 | 9,500 | 6.21 |
| Naphthalene | 11,000 | 1,400 | 7.86 |
| n-Butylbenzene | 6,800 | 860 | 7.91 |
| n-Propylbenzene | 12,000 | 1,800 | 6.67 |
| o-Xylene | 35,000 | 6,400 | 5.47 |
| p-Isopropyltoluene | 7,600 | 990 | 7.68 |
| Toluene | 26,000 | 3,900 | 6.67 |

As can be seen the polyurethane foam exhibits remarkably better performance of detected species (anywhere from 2.44 to 7.91 times better than the PO foam), and also detects some species that are not detected by the polyolefin foam.

Additional comparison testing (efficacy of PU vs. PO) was conducted of an industrial wastewater contaminated with oil and grease. See table 7, below, for results. As before, strips were used. The volume of water, the volume of strips, and the exposure times were the same for both materials. In this case, the reduction of oil and grease was measured, which equates to removal of organic contaminants from (i.e., remediation) of water contaminated with oil or grease.

TABLE 7

| | Oil & Grease | % Oil & Grease |
|---|---|---|
| Oil & Grease/Effluent Testing | (PPM) | Removed |
| Baseline Effluent | 660 | — |
| PU Strips | 190 | 71% |
| PO Strips | 360 | 45% |

As can be seen, the PU removed 71% of the oil and grease, which is a 58% improvement over the removal by the PO strips.

Another comparison test was conducted that equates to removal of surfactants from water. The PU foam and the PO foam were tested with wastewater effluent containing 19,000 ppm of Methylene Blue Active Substances (MBAS). EPA method E 425.1 was used to test the effluent water before exposure to PU and PO foam, and again after exposure. The PU Foam reduced the MBAS by 27% while the PO Foam did not reduce the MBAS.

The testing methods used by certified third party laboratories for the above testing, include but are not limited to: EPA SW8015B, EPA SW7471A, EPA SW6010B, EPA SW8270C, EPA SW8260B, IH-004, ALS Method 8270 (for MCHM), and EPA 1664 (modified) for oil and grease.

A number of implementations have been described. Nevertheless, it will be understood that additional modifications may be made without departing from the scope of the inventive concepts described herein, and, accordingly, other embodiments are within the scope of the following claims.

What is claimed is:

1. A method of removing and detecting the presence of substances from at least one of a body of water and the air, comprising:
placing into the body of water or into the air an open-cell polyurethane foam material;
removing separate portions of the open-cell polyurethane foam material from the water or air at different exposure times after the open-cell polyurethane foam material was placed into the water or air; and
determining the presence in the removed separate portions of one or more substances that were removed from the water or air by the open-cell polyurethane foam material.

2. The method of claim 1, wherein the open-cell polyurethane foam material comprises a plurality of separate structures selected from the group of structures consisting of strips, strips that are longer than a height of a water column, cubes, and small pieces.

3. The method of claim 2, wherein the separate structures are held in place by one or more of an anchor, a weight, a netting, and a container with openings to allow the flow of water therethrough.

4. The method of claim 1, wherein placing the open-cell polyurethane foam material comprises suspending a plurality of separate structures at different levels through a height of a water column.

5. The method of claim 4, wherein placing the open-cell polyurethane foam material further comprises placing a plurality of separate structures at different locations in the body of water.

6. The method of claim 4, wherein placing the open-cell polyurethane foam material further comprises floating a structure at least partially on the surface of the water.

7. The method of claim 1, wherein placing the open-cell polyurethane foam material into the body of water comprises casting the open-cell polyurethane foam material into the water with a fishing rod.

8. The method of claim 1, wherein placing the open-cell polyurethane foam material into the body of water comprises dragging the open-cell polyurethane foam material behind a boat that moves through the water, or floating the open-cell polyurethane foam material on the surface of the water, or coupling the open-cell polyurethane foam material to a dock, or placing the open-cell polyurethane foam material in a bathtub or sink.

9. The method of claim 1, wherein removing separate portions of the open-cell polyurethane foam material from the water or air at different exposure times after the open-cell polyurethane foam material was placed into the water or air, takes place at more than one time over an exposure time of at least eight hours.

10. The method of claim 9, wherein removing separate portions of the open-cell polyurethane foam material from the water or air at different exposure times after the open-cell polyurethane foam material was placed into the water or air, takes place at more than one time over an exposure time of at least one day.

11. The method of claim 1, wherein the substances are selected from the group of substances consisting of oil, diesel range organics, gasoline range organics, drilling fluids, biocides, glutaraldehyde, metals, organometals, metalloids, volatile organic compounds (VOCs), semi-volatile organic compounds (SVOCs), pesticides, polychlorinated biphenyls (PCBs), fertilizers, solvents, human waste, pharmaceuticals, radioactive materials, and components thereof.

12. The method of claim 1, wherein the substances comprise volatile organic compounds (VOCs) and semi-volatile organic compounds (SVOCs).

13. The method of claim 1, wherein the substances comprise a surfactant.

14. The method of claim 1, wherein the substances comprise oils or greases.

15. The method of claim 1, wherein the open-cell foam material comprises a silane-grafted material.

16. The method of claim 1, wherein the open-cell foam material comprises a silane-modified material.

17. A method of removing and/or detecting the presence of substances from water, comprising:
placing into the water an open-cell foam material comprising at least about 70% open cells;
removing one or more separate portions of the open-cell foam material from the water; and
determining the presence in the removed portions of one or more substances that were removed from the water by the open-cell foam material;
wherein the open-cell foam material comprises one or more of ethylene methyl acrylate (EMA), ethylene vinyl acetate (EVA), ethylene-ethyl acrylate (EEA), ethylene-butyl acrylate (EBA), low density polyethylene (LDPE), linear low density polyethylene (LLDPE), very low density polyethylene (VLDPE), high density polyethylene (HDPE), polypropylene (PP), natural rubber, ethylene propylene diene monomer (EPDM), synthetic rubber, chlorinated polyethylene (CPE), olefin block copolymers, ethylene maleic anhydride copolymer, singe site initiated polyolefins, metallocene catalyzed polyolefins, silane-modified polymers (including but not limited to silane grafted, silane functionalized, and silane cross-linked polymers), maleic anhydride grafted polymers, styrene-butadiene-styrene copolymers, polyisoprene, and equivalents and blends thereof.

18. A method of removing and detecting the presence of substances from at least one of a body of water and the air, comprising:
suspending a plurality of separate structures comprising an open-cell foam polyurethane material in the form of strips, strips that are longer than a height of a water column, cubes, and small pieces, at different levels through a height of a water column of the body of water and at different locations in the body of water;
removing separate portions of the structures from the water at different exposure times after the structures were placed into the water; and
determining the presence in the removed separate portions of one or more substances that were removed from the water by the structures, wherein the substances are selected from the group of substances consisting of oil, diesel range organics, gasoline range organics, drilling fluids, biocides, glutaraldehyde, metals, organometals, metalloids, volatile organic compounds (VOCs), semi-volatile organic compounds (SVOCs), surfactants, pesticides, polychlorinated biphenyls (PCBs), fertilizers, solvents, human waste, pharmaceuticals, and components thereof.
